(19) United States
(12) Patent Application Publication
(10) Pub. No.: US 2019/0025242 A1
(43) Pub. Date: Jan. 24, 2019

(54) GENE SEQUENCING CHIP, GENE SEQUENCING APPARATUS, AND GENE SEQUENCING METHOD

(71) Applicants: BOE Technology Group Co., Ltd., Beijing (CN); Beijing BOE Optoelectronics Technology Co., Ltd., Beijing (CN)

(72) Inventors: Fengchun PANG, Beijing (CN); Peizhi CAI, Beijing (CN); Yue GENG, Beijing (CN)

(21) Appl. No.: 15/752,603

(22) PCT Filed: Aug. 18, 2017
(86) PCT No.: PCT/CN2017/098024

§ 371 Date: Feb. 14, 2018

(30) Foreign Application Priority Data
Jan. 3, 2017 (CN) 201710003178.5

(51) Int. Cl. G01N 27/333; C12Q 1/6869; G01N 33/487; G01N 27/327

(52) U.S. Cl. CPC G01N 27/333; G01N 27/3276; G01N 33/48721; C12Q 1/6869

(57) ABSTRACT

The disclosure provides a gene sequencing chip, a gene sequencing apparatus and a gene sequencing method. The gene sequencing chip comprises a substrate; an electrode on the substrate; a signal lead connected with the electrode and configured to input a signal to the electrode and output a signal sensed by the electrode; a first insulation layer on a side of the electrode distal to the substrate; and a baffle, a vertical distance between a surface of the baffle distal to the substrate and the substrate being larger than a vertical distance between a surface of the first insulation layer distal to the substrate and the substrate, and the baffle and the first insulation layer forming a micropore; wherein an orthographic projection of the micropore on the substrate is at least partially overlapped with an orthographic projection of the electrode on the substrate.

### CROSS-REFERENCE TO RELATED APPLICATION

The present application claims priority to Chinese Patent Application NO. 201710003178.5, filed on Jan. 3, 2017, the disclosure of which is hereby incorporated by reference in its entirety.

### TECHNICAL FIELD

The disclosure relates to the field of gene sequencing, particularly, to a gene sequencing chip, a gene sequencing apparatus, and a gene sequencing method.

### BACKGROUND

Gene sequencing technology is a commonly used technology in the modern molecular biology research. As developing from the first generation gene sequencing in 1977, the gene sequencing technology has made a great progress so far, and mainly includes first-generation Sanger sequencing technology, second-generation high-throughput sequencing technology, third-generation single molecule sequencing technology, and fourth-generation nanopore sequencing technology. At present, the second-generation high-throughput sequencing technology is still the mainstream in the market.

The second-generation high-throughput sequencing technology mainly includes sequencing-by-synthesis (Illumina), ion semiconductor sequencing from Thermo Fisher, sequencing by ligation, pyrosequencing by Roche, etc.

The ion semiconductor sequencing method includes the following steps: at first, preparing a library, breaking deoxyribonucleic acid (DNA) to be sequenced into segments by nebulization, and adding different adaptors at two ends of each segment to construct a single-stranded-DNA library; secondly, performing emulsion amplification, so that these single-stranded DNA molecules are combined onto magnetic beads with a diameter of about 20 μm and covered with water and oil, and incubate and undergo annealing on the magnetic beads. By amplification, each segment is amplified by about one million times so as to reach the DNA quantity required for subsequent sequencing. Finally, sequencing is performed as follows: the magnetic beads are put into micropores, nucleotide molecules consecutively flow one by one through the micropores of the chip during the sequencing, and if a deoxynucleotide is complementary to a DNA molecule in a specific micropore, this deoxynucleotide is synthesized into the DNA molecule and hydrogen ions are released, so that the pH value of the solution in the mircropore is changed. Once the change in pH value is detected by an ion sensor, chemical information is converted into digital electronic information.

However, in the above method, an ion sensor needs to be formed below the micropore. The ion sensor is manufactured by a CMOS process and includes two MOSFETs (Metal-Oxide-Semiconductor Field-Effect Transistors) and one ISFET (Ion-Sensitive Field-Effect Transistor). During manufacturing of an existing gene sequencing chip, multiple processes of masking, exposure, developing and etching are required, resulting in a complicated manufacturing process and a high cost.

### SUMMARY

In order to solve the above problem in the prior art, the disclosure provides a gene sequencing chip which need no field effect transistor, and has a simple manufacturing process, thereby significantly reducing the manufacturing difficulty and the cost. The disclosure further relates to a gene sequencing apparatus including the gene sequencing chip.

Furthermore, the disclosure provides a gene sequencing method which utilizes the gene sequencing chip and can perform gene sequencing easily and conveniently.

The gene sequencing chip provided by the disclosure includes a substrate; an electrode on the substrate; a signal lead connected with the electrode and configured to input a signal to the electrode and output a signal sensed by the electrode; a first insulation layer on a side of the electrode distal to the substrate; and a baffle, a vertical distance between a surface of the baffle distal to the substrate and the substrate being larger than a vertical distance between a surface of the first insulation layer distal to the substrate and the substrate, and the baffle and the first insulation layer forming a micropore; wherein an orthographic projection of the micropore on the substrate is at least partially overlapped with an orthographic projection of the electrode on the substrate.

According to an embodiment of the disclosure, the gene sequencing chip further includes an ion-sensitive film in contact with the first insulation layer and on the side of the first insulation layer distal to the substrate. The ion-sensitive film can make change in the signal sensored by the electrode become more evident.

According to an embodiment of the disclosure, a material of the ion-sensitive film is Si₃N₄. The ion-sensitive film made of Si₃N₄ is quite sensitive to hydrogen ions.

According to an embodiment of the disclosure, the first insulation layer covers the substrate, and the baffle is on the side of the first insulation layer distal to the substrate. Alternatively, an orthographic projection of the first insulation layer on the substrate completely coincides with the orthographic projection of the micropore on the substrate.

According to an embodiment of the disclosure, the first insulation layer and the baffle are formed as an integral structure by a single material.

According to an embodiment of the disclosure, the orthographic projection of the micropore on the substrate completely coincides with the orthographic projection of the electrode on the substrate.

According to an embodiment of the disclosure, the signal lead and the electrode are arranged in one layer.

According to an embodiment of the disclosure, a second insulation layer is provided between the signal lead and the electrode, and the signal lead and the electrode are connected with each other through a via in the second insulation layer.

According to an embodiment of the disclosure, the electrode and the signal lead are made of a metal such as Mo, Al or Cu. The baffle, the first insulation and the second insulation are made of silicon nitride or silicon oxide. The signal lead may be arranged at one side or both sides of the substrate.

The disclosure further provides a gene sequencing apparatus comprising the gene sequencing chip according to the disclosure.

According to an embodiment of the disclosure, the gene sequencing apparatus further includes a detecting chip configured to send a signal to the electrode through the signal lead and receive a signal sensed by the electrode through the signal lead.

The disclosure further provides a gene sequencing method using the gene sequencing chip according to the disclosure, including steps of: adding a DNA microbead containing a DNA strand into the micropore of the gene sequencing chip to perform polymerase chain reaction (PCR) amplification; adding four types of deoxy-ribonucleoside triphosphate (dNTP) into the micropore in sequence; applying a signal to the electrode of the gene sequencing chip through the signal lead of the gene sequencing chip and detecting whether a value of the signal sensed by the electrode changes, by the detecting chip; and determining a type of a base on the DNA strand according to the type of the dNTP added at the time when the value of the signal changes.

According to an embodiment of the disclosure, the dNTP is a reversibly terminated dNTP, and the gene sequencing method further includes: cleaning the reversibly terminated dNTP added into the micropore, and adding a mercapto reagent.

### BRIEF DESCRIPTION OF THE FIGURES

Drawings, which constitute a part of the description, are provided to explain the present disclosure in conjunction with the following specific implementations so as to provide a further understanding, instead of a limitation, of the present disclosure. In the drawings:

FIG. 1 shows a top view of a gene sequencing chip according to an embodiment of the disclosure;

FIGS. 2 to 4 are cross-sectional views taken along line A-A′ in FIG. 1 of the gene sequencing chip according to the embodiment of the disclosure;

FIG. 5 shows a top view of a gene sequencing chip according to another embodiment of the disclosure;

FIGS. 6 to 8 are cross-sectional views taken along line A-A′ in FIG. 5 of the gene sequencing chip according to the other embodiment of the disclosure; and

FIG. 9 is a flow chart showing a gene sequencing method according to an embodiment of the disclosure.

### DETAILED DESCRIPTION

In order that the objective, the solutions and the advantageous of embodiments of the disclosure are more apparent, the technical solutions of the embodiments of the disclosure will be described clearly and thoroughly below in conjunction with the drawings of the embodiments of the disclosure. Obviously, the embodiments as described are some of the embodiments of the disclosure instead of all embodiments. All other embodiments that can be obtained by an ordinary person skilled in the art based on the described embodiments of the disclosure also fall into the protective scope of the disclosure.

Unless otherwise defined, technical terms or scientific terms as used herein should have common meanings as understood by an ordinary person skilled in the art to which the disclosure belongs. The words “first”, “second” and the like as used in the specification and claims of the disclosure do not indicate any sequence, quantity or importance, but are simply used for distinguishing different components. Also, the word “a” or “an” or the like does not indicate a limitation of quantity, but means at least one. The expression “connecting” or “connecting with” or the like is not limited to a physical or a mechanical connection, but may include an electrical connection, no matter it is direct or indirect. The expressions “above”, “below”, “on the left”, “on the right” and the like are used for representing a relative positional relationship only, and the relative positional relationship will be changed accordingly when absolute positions of the described objects are changed.

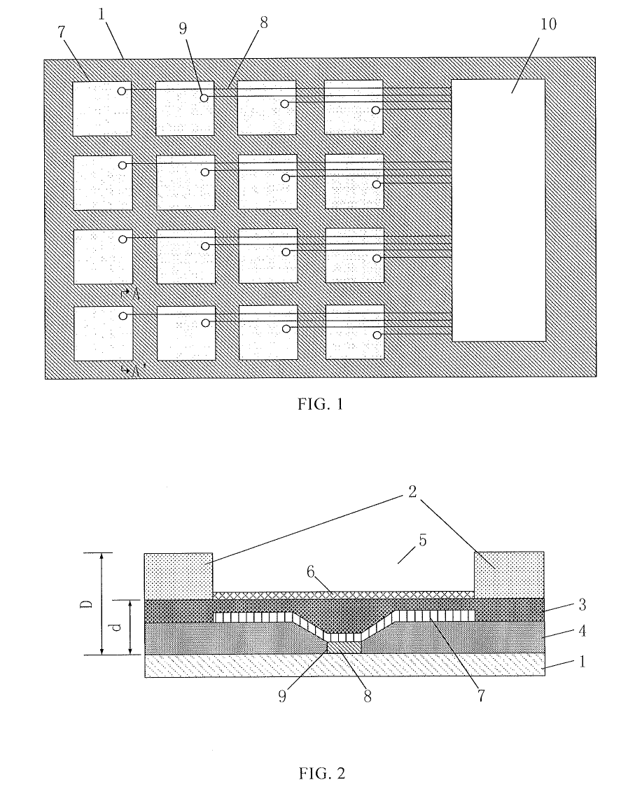

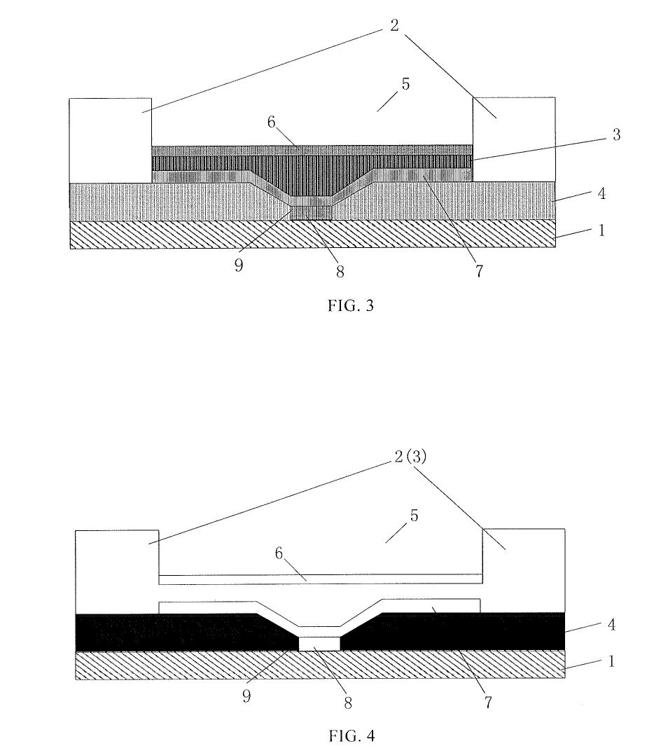

FIG. 1 shows a top view of a gene sequencing chip according to an embodiment of the disclosure. FIG. 2 is a cross sectional view taken along line A-A′ in FIG. 1 of the gene sequencing chip according to the embodiment of the disclosure.

Referring to FIG. 1, the gene sequencing chip according to the embodiment of the disclosure includes a substrate 1, on which electrodes 7 and signal leads 8 are provides. Each signal lead 8 is connected with one of the electrodes 7 and is configured to input a signal to the electrode 7 and output a signal sensed by the electrode 7 to a signal transmitting terminal. The gene sequencing chip further includes a first insulation layer 3 and baffles 2. The first insulation layer 3 is located on a side of the electrodes 7 distal to the substrate 1, and a vertical distance D between a surface of each baffle 2 distal to the substrate 1 and the substrate 1 is larger than a vertical distance d between a surface of the first insulation layer 3 distal to the substrate 1 and the substrate 1. The baffles 2 and the first insulation layer 3 form micropores 5. An orthographic projection of each micropore 5 on the substrate 1 is at least partially overlapped with an orthographic projection of one of the electrodes 7 on the substrate 1.

The gene sequencing chip of the disclosure does not need a field effect transistor, and thus has a simply manufacturing process, thereby lowering manufacturing difficulty and the cost. Moreover, according to a gene sequencing method of the disclosure, the gene sequencing can be performed easily and conveniently.

It should be understood that the surfaces of the first insulation layer 3 and the baffles 2 may be uneven. For example, as long as there is a distance difference between the lowest point on the surface of the baffle 2 distal to the substrate 1 and the highest point on the surface of the first insulation layer 3 distal to the substrate 1, the micropores can be formed.

The baffles 2 may be made of an insulation material. The material for forming the baffles 2 may include (but not limited to) silicon oxide, silicon nitride, silicon oxynitride, insulation resin material, or the like. The electrodes 7 and the signal leads 8 may be made of a metal such as Mo, Al or Cu.

In an embodiment of the present disclosure, the orthographic projection of each micropore 5 on the substrate 1 completely coincides with the orthographic projection of one of the electrodes 7 on the substrate 1. When reaction occurs in the micropore, the electrode 7 can sense a signal change caused by the reaction occurring in the whole micropore 5, which further improves the detecting sensitivity.

Optionally, the gene sequencing chip may further include a detecting chip 10. One end of each signal lead 8 is connected to one of the electrodes 7, and the other end thereof is connected to the detecting chip 10 serving as the signal transmitting terminal. The signal leads 8 are used for inputting a signal from the detecting chip 10 to the electrodes 7, and outputting signals sensed by the electrodes 7 to the detecting chip 10. Although it is illustrated in FIG. 1 that the detecting chip 10 is formed on the substrate 1, the solution of the disclosure is not limited thereto. The detecting chip 10 may be formed as an element separate from the gene sequencing chip of the disclosure. The detecting chip 10 in the disclosure sends a voltage pulse signal to the electrodes 7 through the signal leads 8, and detects whether a value of the signal sensed by each electrode 7 is changed.

Referring to FIG. 2, the electrode 7 and the signal lead 8 are arranged in different layers, and the electrode 7 is connected to the signal lead 8 through an via 9 in the second insulation layer 4. The first insulation layer 3 is an integral layer, i.e., it totally covers the surface, on which the electrodes 7 and the leads 8 are formed, of the substrate 1. The baffle 2 is provided on the first insulation layer 3, and the vertical distance D between the surface of the baffle 2 distal to the substrate 1 and the substrate 1 is larger than the vertical distance d between the surface of the first insulation layer 3 distal to the substrate 1 and the substrate 1. Thus, the micropore 5 is formed by the baffle 2 and the first insulation layer 3. The orthographic projection of the micropore 5 on the substrate 1 may be at least partially overlapped with the orthographic projection of the electrode 7 on the substrate 1. The material of the second insulation layer 4 may be silicon nitride or silicon oxide or the like. Since the electrode 7 and the signal lead 8 are arranged in different layers, it is unnecessary to reserve a wiring space between adjacent electrodes 7 for the signal lead 8. Therefore, the electrodes 7 on the substrate 1 can be arranged more compactly, so that the number of the electrodes 7 on the substrate 1 can be increased.

According to an embodiment of the disclosure, an ion-sensitive film 6 is provided in each micropore 5. The ion-sensitive film 6 is in contact with the first insulation layer 3 and located on a side of the first insulation layer 3 distal to the substrate 1.

When complementary base pairing occurs in a micropore 5, hydrogen ions may be released, and Nernstian potential is induced at the surface of the ion-sensitive film 6, which has an effect on the voltage pulse signal on the electrode 7. The Nernstian potential sensed by the ion-sensitive film 6 can make the change in the signal sensed by the electrode more evident.

According to an embodiment of the disclosure, a material of the ion-sensitive film 6 is Si₃N₄. The ion-sensitive film 6 made of Si₃N₄ is more sensitive to the hydrogen ions.

FIGS. 3 and 4 show variant examples according to an embodiment of the disclosure. Compared with the embodiment as shown in FIG. 2, the baffle 2 and the first insulation layer 3 are arranged in a different manner.

Different from the arrangement that the first insulation layer 3 is formed as an integral layer as shown in FIG. 2, as shown in FIG. 3, the orthographic projection of the first insulation layer 3 on the substrate 1 completely coincides with the orthographic projection of the micropore 5 on the substrate 1.

As shown in FIG. 4, the baffle 2 and the first insulation layer 3 may be formed integrally using one material, which helps to further reduce the manufacturing difficulty and the cost.

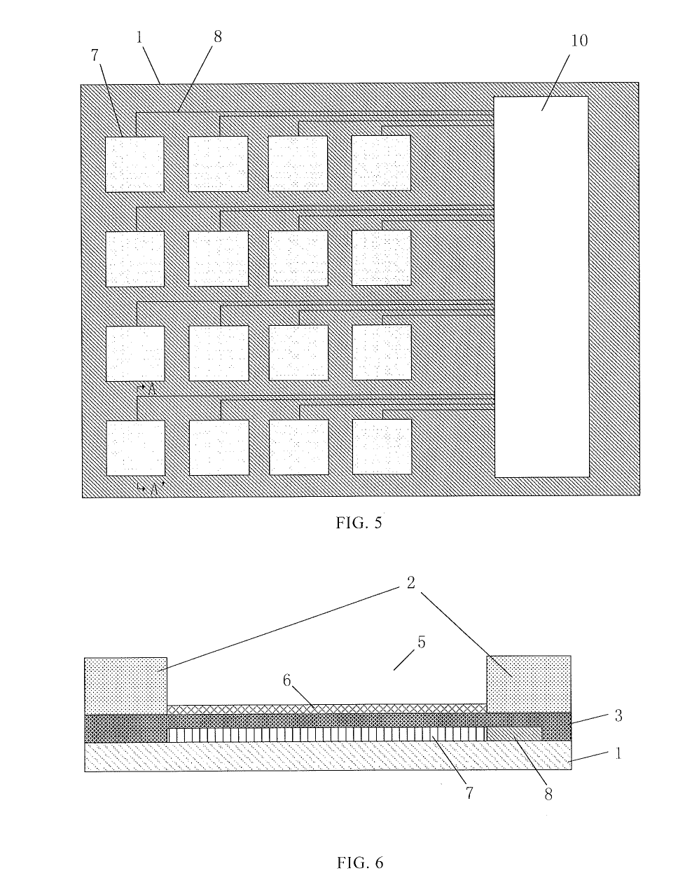

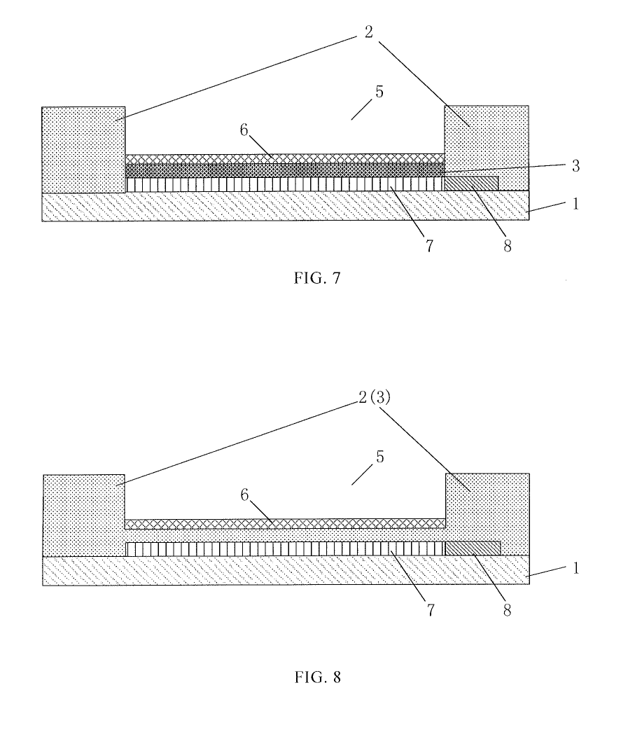

FIG. 5 shows a top view of a gene sequencing chip according to another embodiment of the disclosure. FIG. 6 is a cross sectional view taken along line A-A′ in FIG. 5 of the gene sequencing chip according to the other embodiment of the disclosure. Thereinafter, descriptions will focus on the differences between the gene sequencing chip of the present embodiment and the gene sequencing chips of the above embodiments. For simplicity, those similar to the above embodiments will be omitted, and like reference numerals represent like components.

Referring to FIGS. 5 and 6, the signal leads 8 and the electrodes 7 may be arranged in a single layer. The electrodes 7 and the signal leads 8 may be manufactured simultaneously so as to further reduce the manufacturing difficulty and the cost.

FIGS. 7 and 8 show variant examples according to an embodiment of the disclosure. Compared with the embodiment of FIG. 6, the baffle 2 and the first insulation layer 3 are arranged in a different manner.

Similar to the embodiment shown in FIG. 3, the orthographic projection of the first insulation layer 3 on the substrate 1 completely coincides with the orthographic projection of the micropore 5 on the substrate 1, as shown in FIG. 7.

Similar to the embodiment shown in FIG. 4, the baffle 2 and the first insulation layer 3 may be formed integrally using one material so as to further reduce the manufacturing difficulty and the cost, as shown in FIG. 8.

It should be appreciated that, the ion-sensitive film 6 as shown in FIGS. 2 to 4 and FIGS. 6 to 8 are not necessary. According to embodiments of the disclosure, in the absence of the ion-sensitive film, hydrogen ions will be released if complementary base pairing occurs in a micropore 5. The released hydrogen ions may have an effect on the voltage pulse signal on the electrode 7, and thus the type of a base on a DNA strand may be determined according to deoxyribonucleoside triphosphate (dNTP) which is added at the time when the signal value changes.

It can be seen that the gene sequencing chip according to the disclosure can achieve gene sequencing according to the change of the voltage pulse signal on the electrode 7 without using a field effect transistor, thereby reducing the manufacturing difficulty and the cost.

At least one embodiment of the disclosure provides a gene sequencing apparatus including the above gene sequencing chip.

At least one embodiment of the disclosure provides a gene sequencing apparatus including the above gene sequencing chip and the detecting chip 10. The detecting chip 10 is used for sending a signal to the electrodes 7 through the signal leads 8 and receiving signals sensed by the electrodes 7 through the signal leads 8.

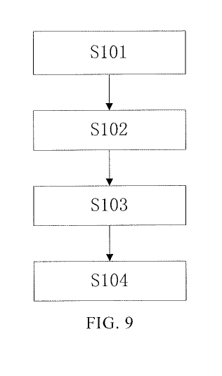

FIG. 9 shows a flow chart of a gene sequencing method according to an embodiment of the disclosure.

The gene sequencing method using the gene sequencing apparatus according to the disclosure will be described below with reference to FIGS. 1 and 9.

The gene sequencing apparatus according to an embodiment of the disclosure may include the gene sequencing chip and the detecting chip according to the disclosure. As shown in FIG. 9, the gene sequencing method using the gene sequencing apparatus according to the disclosure includes the following steps:

S101: placing a DNA microbead containing a DNA strand into one of the micropores 5 of the gene sequencing chip to perform PCR amplification;

S102: adding four types of dNTP into the micropore 5 in sequence;

S103: inputting a signal to the electrode 7 of the gene sequencing chip through the signal lead 8 of the gene sequencing chip, and detecting whether a value of the signal sensed by the electrode 7 changes; and

S104: determining the type of a base on the DNA strand according to the dNTP added at the time when the signal value changes.

According to an embodiment of the disclosure, the dNTP used in step S102 is a reversibly terminated dNTP, which may include, for example, reversibly terminated deoxyadenosine triphosphate (dATP), reversibly terminated deoxy-thymidine triphosphate (dTTP), reversibly terminated deoxycytidine triphosphate (dCTP), and reversibly terminated deoxyguanosine triphosphate (dGTP).

In at least one embodiment of the disclosure, sending a voltage pulse signal to the electrode 7 and receiving a sensed signal from the electrode 7 may be performed by the monitoring chip 10 in a time-division manner. That is, firstly the voltage pulse signal is input to the electrode 7 through the signal lead 8, and then the signal sensed by the electrode 7 is received also through the signal lead 8. When the dNTP in the micropore 5 is synthesized into the DNA molecule, hydrogen ions may be released. The hydrogen ions may have an effect on the voltage pulse signal on the electrode 7. According to an embodiment of the disclosure, if an ion-sensitive film 6 is provided in the micropore 5, Nernstian potential may be induced on the surface of the ion-sensitive film 6 by the hydrogen ions. The Nernstian potential may also have an effect on the voltage pulse signal on the electrode 7. The type of the base on the DNA strand can be determined according to the dNTP added at the time when the signal value changes.

Specifically, at the time when the value of the signal sensed by the electrode 7 changes, if the dNTP added in the micropore 5 is dATP, then the base on the DNA strand to be sequenced is thymine; if the dNTP added in the micropore 5 is dTTP, then the base on the DNA strand to be sequenced is adenine; if the dNTP added in the micropore 5 is dCTP, then the base on the DNA strand to be sequenced is guanine; and if the dNTP added in the micropore 5 is dGTP, then the base on the DNA strand to be sequenced is cytosine.

After detection of the type of the base at one position on DNA is completed, the reversibly terminated dNTP added into the micropore 5 is cleaned, and a mercapto reagent is added. Unlike a common dNTP, a common reversibly terminated dNTP has its 3′ end linked to an azide group, and a phosphodiester bond cannot be formed during DNA synthesis, and thus the DNA synthesis interrupts. However, if the mercapto reagent is added, the azide group will be broken, and a hydroxyl will be formed at the same position. After adding the mercapto reagent, the detection of the types of the bases on subsequent positions can be continued.

Therefore, the types of the bases on the DNA strand can be determined by simply judging the signal change and determining the type of the added dNTP. This is convenient for the gene sequencing method.

Foregoing are preferable implementations of the disclosure. It should be noted that an ordinary person skilled in the art may made various improvements and modifications without departing from the spirit and essence of the present invention. The protective scope of the present invention is defined by the claims.

1. A gene sequencing chip, comprising:
a substrate;
an electrode on the substrate;
a signal lead connected with the electrode and configured to input a signal to the electrode and output a signal sensed by the electrode;
a first insulation layer on a side of the electrode distal to the substrate; and
a baffle, a vertical distance between a surface of the baffle distal to the substrate and the substrate being larger than a vertical distance between a surface of the first insulation layer distal to the substrate and the substrate, and the baffle and the first insulation layer forming a micropore;
wherein an orthographic projection of the micropore on the substrate is at least partially overlapped with an orthographic projection of the electrode on the substrate.

2. The gene sequencing chip of claim 1, further comprising an ion-sensitive film in contact with the first insulation layer and on a side of the first insulation layer distal to the substrate.

3. The gene sequencing chip of claim 2, wherein a material of the ion-sensitive film is Si₃N₄.

4. The gene sequencing chip of claim 1, wherein the first insulation layer covers the substrate, and the baffle is on a side of the first insulation layer distal to the substrate.

5. The gene sequencing chip of claim 1, wherein an orthographic projection of the first insulation layer on the substrate completely coincides with the orthographic projection of the micropore on the substrate.

6. The gene sequencing chip of claim 4, wherein the first insulation layer and the baffle are formed as an integral structure by one material.

7. The gene sequencing chip of claim 1, wherein the orthographic projection of the micropore on the substrate completely coincides with the orthographic projection of the electrode on the substrate.

8. The gene sequencing chip of claim 1, wherein the signal lead and the electrode are arranged in one layer.

9. The gene sequencing chip of claim 1, further comprising a second insulation layer between the signal lead and the electrode, wherein the signal lead and the electrode are connected with each other through a via in the second insulation layer.

10. A gene sequencing apparatus, comprising a gene sequencing chip including:
a substrate;
an electrode on the substrate;
a signal lead connected with the electrode and configured to input a signal to the electrode and output a signal sensed by the electrode;
a first insulation layer on a side of the electrode distal to the substrate; and
a baffle, a vertical distance between a surface of the baffle distal to the substrate and the substrate being larger than a vertical distance between a surface of the first insulation layer distal to the substrate and the substrate, and the baffle and the first insulation layer forming a micropore;
wherein an orthographic projection of the micropore on the substrate is at least partially overlapped with an orthographic projection of the electrode on the substrate.

11. The apparatus of claim 10, further comprising a detecting chip configured to send a signal to the electrode through the signal lead and receive a signal sensed by the electrode through the signal lead.

12. A gene sequencing method, the gene sequencing method using a gene sequencing chip comprising:
a substrate;
an electrode on the substrate;
a signal lead connected with the electrode and configured to input a signal to the electrode and output a signal sensed by the electrode;
a first insulation layer on a side of the electrode distal to the substrate; and
a baffle, a vertical distance between a surface of the baffle distal to the substrate and the substrate being larger than a vertical distance between a surface of the first insulation layer distal to the substrate and the substrate, and the baffle and the first insulation layer forming a micropore;
wherein an orthographic projection of the micropore on the substrate is at least partially overlapped with an orthographic projection of the electrode on the substrate, and
the method comprising steps of:
adding a deoxyribonucleic acid (DNA) microbead containing a DNA strand into the micropore of the gene sequencing chip to perform polymerase chain reaction (PCR) amplification;
adding four types of deoxy-ribonucleoside triphosphate (dNTP) into the micropore in sequence;
applying a signal to the electrode through the signal lead of the gene sequencing chip, and detecting whether a value of the signal sensed by the electrode changes; and
determining a type of a base on the DNA strand according to the dNTP added at the time when the value of the signal changes.

13. The gene sequencing method of claim 12, wherein the dNTP is a reversibly terminated dNTP, and the gene sequencing method further comprises:
cleaning the reversibly terminated dNTP added into the micropore, and adding a mercapto reagent.

14. The gene sequencing chip of claim 5, wherein the first insulation layer and the baffle are formed as an integral structure by one material.

15. The apparatus of claim 10, wherein the gene sequencing chip further comprises an ion-sensitive film in contact with the first insulation layer and on a side of the first insulation layer distal to the substrate.

16. The apparatus of claim 15, wherein a material of the ion-sensitive film is Si₃N₄.

17. The apparatus of claim 10, wherein the first insulation layer covers the substrate, and the baffle is on a side of the first insulation layer distal to the substrate.

18. The apparatus of claim 10, wherein an orthographic projection of the first insulation layer on the substrate completely coincides with the orthographic projection of the micropore on the substrate.

19. The apparatus of claim 17, wherein the first insulation layer and the baffle are formed as an integral structure by one material.

20. The apparatus of claim 10, wherein the orthographic projection of the micropore on the substrate completely coincides with the orthographic projection of the electrode on the substrate.
